(19) United States
(12) Patent Application Publication
(10) Pub. No.: US 2023/0043647 A1
(43) Pub. Date: Feb. 9, 2023

(54) FASTENER AND METHOD

(71) Applicant: Jeffrey HARRISON, Huntsville, UT (US)

(72) Inventor: Jeffrey HARRISON, Huntsville, UT (US)

(21) Appl. No.: 17/881,153

(22) Filed: Aug. 4, 2022

Related U.S. Application Data
(60) Provisional application No. 63/229,271, filed on Aug. 4, 2021.

(51) Int. Cl. A61F 2/08

(52) U.S. Cl. CPC A61F 2/0811; A61F 2002/0858

(57) ABSTRACT

A fastener is disclosed for anchoring a tether within a body of a patient. The fastener includes a one-way tissue engagement feature having an opening that receives a length of a tether and permits the length of the tether to pass through the opening in a first direction and restricts movement of the tether in a second direction opposite the first direction. The fastener also includes an engagement feature configured to maintain contact between the fastener and a bone, or other tissue, of the patient in response to tension applied to the tether in the second direction to keep the fastener in place relative to the bone.

### CROSS-REFERENCE TO RELATED APPLICATIONS

This application claims the benefit of U.S. Provisional Application No. 63/229,271, filed Aug. 4, 2021, entitled FIXATION DEVICE AND METHOD, which is hereby incorporated by reference in its entirety.

### TECHNICAL FIELD

The present disclosure relates to surgical devices, systems, instruments, and methods. More specifically, the present disclosure relates to tissue fixation, and methods of designing and/or using the same.

### BACKGROUND

Various surgical procedures in people and animals include a fixation step, procedure, use of a device (also referred to as a fixator or fastener), or the like. The fixation feature, or function, may be between two pieces of bone, soft tissue and bone, soft tissue and hard tissue, soft tissue and soft tissue, and/or a combination of these. Unfortunately, many known fixation techniques, steps, procedures, devices, or components are complicated, involve multiple parts, are not easy to revise, become loose over time, are difficult to tension, and have other limitations.

### SUMMARY

The various apparatus, devices, systems, and/or methods of the present disclosure have been developed in response to the present state of the art, and in particular, in response to the problems and needs in the art that have not yet been fully solved by currently available technology. One general aspect of the present disclosure can include a fastener for anchoring a tether within a body of a patient. The fastener includes a one-way tissue engagement feature having an opening that receives a length of a tether and permits the length of the tether to pass through the opening in a first direction and restricts movement of the tether in a second direction opposite the first direction; and an engagement feature configured to maintain contact between the fastener and a bone, or other tissue, of the patient in response to tension applied to the tether in the second direction. The tension applied to the tether may keep the fastener in place relative to the bone, or other tissue.

Implementations may include one or more of the following features. The fastener may include a body that circumscribes the one-way tissue engagement feature and where the engagement feature extends from the body and faces at least partially toward the second direction to contact part of the bone of the patient. The body is tube shaped and the engagement feature may include a flange circumscribing the body. The fastener may include a distal end and a proximal end; the opening extends between the distal end and the proximal end; and the engagement feature is proximal to the proximal end and the first direction starts at the distal end and continues to the proximal end. The one-way tissue engagement feature may include a set of teeth that extend into the opening, the set of teeth configured to engage the tether within the opening such that the tether can move through the opening in the first direction and not move through the opening in the second direction. Each tooth of the set of teeth may include a base connected to an interior wall of the opening and a tip, each tooth oriented such that the tip is displaced in the first direction relative to the base. The teeth of the set of teeth are pliable and configured to bend toward the first direction as the tether is moved through the opening in the first direction. A tip of at least one tooth of the set of teeth may include a tapered edge; and the tip extends into the tether in response to a tension force on the tether in the second direction. The tip of at least one tooth of the set of teeth remains within the opening.

One general aspect of the present disclosure can include a one-way tether lock for anchoring a tissue graft within a bone tunnel. The one-way tether lock includes a cylindrical body having a proximal end, a distal end, and an opening that extends from the distal end to the proximal end; and a set of teeth that extend into the opening, the set of teeth configured to permit passage of a tissue graft from the distal end toward the proximal end and constrain passage of the tissue graft from the proximal end toward the distal end.

Implementations may include one or more of the following features. The one-way tether lock may include a stop configured to constrain movement of the one-way tether lock from a proximal end of a bone tunnel toward a distal end of the bone tunnel. The stop connects to the cylindrical body at the proximal end. The stop may include a lip that circumscribes the cylindrical body, the lip sized to contact a surface of bone around the bone tunnel that accepts the one-way tether lock. The bone tunnel extends from a surface of a bone at first angle not perpendicular to the surface and the lip extends from the cylindrical body at a second angle that corresponds to the first angle and a first diameter of the bone tunnel is larger than a second diameter of the cylindrical body and smaller than a third diameter of the lip. The cylindrical body may include a first diameter sized to fit within a bone tunnel configured to accept the one-way tether lock and the stop may include part of the cylindrical body having a second diameter greater than the first diameter. Teeth of the set of teeth include a base at one end and an edge at an opposite end and where one or more teeth of the set of teeth connect to the cylindrical body at the base and extend to point the edge towards the proximal end. Teeth of the set of teeth define an initial diameter passage in the opening and the teeth are pliable and bend towards the proximal end to define a second diameter passage having a diameter greater than the initial diameter passage.

One general aspect of the present disclosure can include a method for anchoring a tissue graft within a bone tunnel of a patient. The method includes forming a bone tunnel in a bone of the patient; deploying a tissue graft within the bone tunnel; fixing a first end of the tissue graft relative to a first end of the bone tunnel; threading a second end of the tissue graft through an opening in a cortical graft anchor; and sliding the tissue graft through the opening in a first direction such that teeth within the opening of the cortical graft anchor engage the tissue graft to prevent motion of the tissue graft, in a second direction opposite the first direction; and engaging a stop of the cortical graft anchor with a second end of the bone tunnel in response to tension in the tissue graft pulling the cortical graft anchor toward the bone tunnel.

Implementations may include one or more of the following features. The method may include forming a cutout around a second end of the bone tunnel, the cutout sized to accept a stop of the cortical graft anchor; and seating the stop with the cutout at the second end of the bone tunnel. The method may include tensioning the tissue graft by pulling the tissue graft through the opening and away from the bone tunnel.

### BRIEF DESCRIPTION OF THE DRAWINGS

The advantages, nature, and additional features of exemplary embodiments of the disclosure will become more fully apparent from the following description and appended claims, taken in conjunction with the accompanying drawings. Understanding that these drawings depict only exemplary embodiments and are, therefore, not to be considered limiting of the disclosure's scope, the exemplary embodiments of the disclosure will be described with additional specificity and detail through use of the accompanying drawings in which:

FIG. 1A is a perspective view of a fastener, according to one embodiment.

FIG. 1B is a side view of a fastener, according to one embodiment.

FIG. 1C is a side view of a fastener, according to one embodiment.

FIG. 2A is a perspective view of a fastener, according to one embodiment.

FIG. 2B is a perspective view of the fastener of FIG. 2A from a first end, according to one embodiment.

FIG. 2C is a perspective view of the fastener of FIG. 2A from a second end, according to one embodiment.

FIG. 2D is an end view of the fastener of FIG. 2A from the first end, according to one embodiment.

FIG. 3A is a cross-sectional view of a fastener taken along a longitudinal axis of the fastener, according to one embodiment.

FIG. 3B is an end view from a distal end of a fastener, according to one embodiment.

FIG. 4 is a cross-sectional view of a fastener taken along a longitudinal axis of the fastener positioned within a bone tunnel of a bone, according to one embodiment.

FIG. 5A is a side perspective view, end perspective view, opposite end perspective view, and a side perspective view of a fastener having a circular longitudinal cross-section in four different orientations, according to one embodiment.

FIG. 5B is a side perspective view, end perspective view, opposite end perspective view, and a side perspective view of a fastener having an elliptical longitudinal cross-section in four different orientations, according to one embodiment.

FIG. 5C is a side perspective view, end perspective view, opposite end perspective view, and a side perspective view of a fastener having a circular longitudinal cross-section and a non-uniform diameter in four different orientations, according to one embodiment.

FIG. 5D is a side perspective view, end perspective view, opposite end perspective view, and a side perspective view of a fastener having a square longitudinal cross-section in four different orientations, according to one embodiment.

FIG. 5E is an end view of a one-way tissue engagement feature, according to one embodiment.

FIG. 5F is an end view of a one-way tissue engagement feature, according to one embodiment.

FIG. 6 illustrates one example of a method for securing tissue within, or to, a body of a patient, according to one embodiment.

FIGS. 7A-7D illustrate cross-sectional views of different stages of practicing a method according to one embodiment of the present disclosure.

### DETAILED DESCRIPTION

Exemplary embodiments of the disclosure will be best understood by reference to the drawings, wherein like parts are designated by like numerals throughout. It will be readily understood that the components, as generally described and illustrated in the Figures herein, could be arranged and designed in a wide variety of different configurations. Thus, the following more detailed description of the embodiments of the apparatus, system, and method is not intended to limit the scope of the invention, as claimed, but is merely representative of exemplary embodiments of the technology.

Standard medical planes of reference and descriptive terminology are employed in this disclosure. While these terms are commonly used to refer to the human body, certain terms are applicable to physical objects in general. A standard system of three mutually perpendicular reference planes is employed. A sagittal plane divides a body into right and left portions. A coronal plane divides a body into anterior and posterior portions. A transverse plane divides a body into superior and inferior portions.

A mid-sagittal, mid-coronal, or mid-transverse plane divides a body into equal portions, which may be bilaterally symmetric. The intersection of the sagittal and coronal planes defines a superior-inferior or cephalad-caudal axis. The intersection of the sagittal and transverse planes defines an anterior-posterior axis. The intersection of the coronal and transverse planes defines a medial-lateral axis. The superior-inferior or cephalad-caudal axis, the anterior-posterior axis, and the medial-lateral axis are mutually perpendicular.

Anterior means toward the front of a body. Posterior means toward the back of a body. Superior or cephalad means toward the head. Inferior or caudal means toward the feet or tail. Medial means toward the midline of a body, particularly toward a plane of bilateral symmetry of the body. Lateral means away from the midline of a body or away from a plane of bilateral symmetry of the body. Axial means toward a central axis of a body. Abaxial means away from a central axis of a body. Ipsilateral means on the same side of the body. Contralateral means on the opposite side of the body from the side which has a particular condition or structure.

Proximal means toward the trunk of the body. Proximal may also mean toward a user, viewer, or operator. Distal means away from the trunk. Distal may also mean away from a user, viewer, or operator. Dorsal means toward the top of the foot or other body structure. Plantar means toward the sole of the foot or toward the bottom of the body structure. Antegrade means forward moving from a proximal location/position to a distal location/position or moving in a forward direction. Retrograde means backward moving from a distal location/position to a proximal location/position or moving in a backwards direction. Sagittal refers to a midline of a patient's anatomy, which divides the body into left or right halves. The sagittal plane may be in the center of the body, splitting it into two halves. Prone means a body of a person lying face down. Supine means a body of a person lying face up.

As used herein, a “deploy” or “deployment” refers to an act, action, process, system, method, means, or apparatus for inserting an implant or prosthesis into a part, body part, and/or patient. “Deploy” or “deployment” can also refer to an act, action, process, system, method, means, or apparatus for placing something into therapeutic use. A device, system, component, medication, drug, compound, or nutrient may be deployed by a human operator, a mechanical device, an automated system, a computer system or program, a robotic system, or the like.

The present disclosure discloses surgical devices, systems, and/or methods for fixation of tissue within a body of a patient. Known fixators and/or fasteners, methods, or steps are limited. Fasteners may be aperture fixation (e.g., interference screws) or suspensory fixation (e.g., EZLoc, WasherLoc, Toggleloc plus EZloc, and the like; cortical: Endo buttons, staples, screw posts; cancellous: transfixion pins, Bio-TransFix, Aperfix, and the like). A simple, easy fastener that facilitates setting a tension in soft tissue is needed.

The present disclosure discloses a fastener and/or method for fixation of tissue that is quick and easy to deploy, low profile, readily revisable, requires no special knots or supporting accessories or equipment, and provides a strong fixation that is secure and provides the desired level of fixation in view of tensions experienced by tissue in a joint such as a knee.

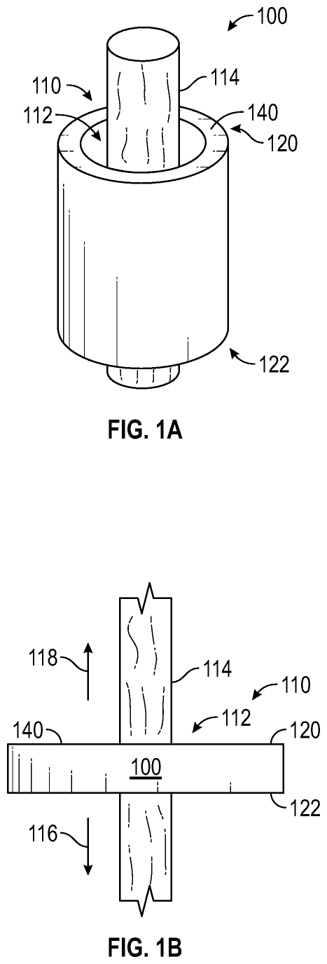

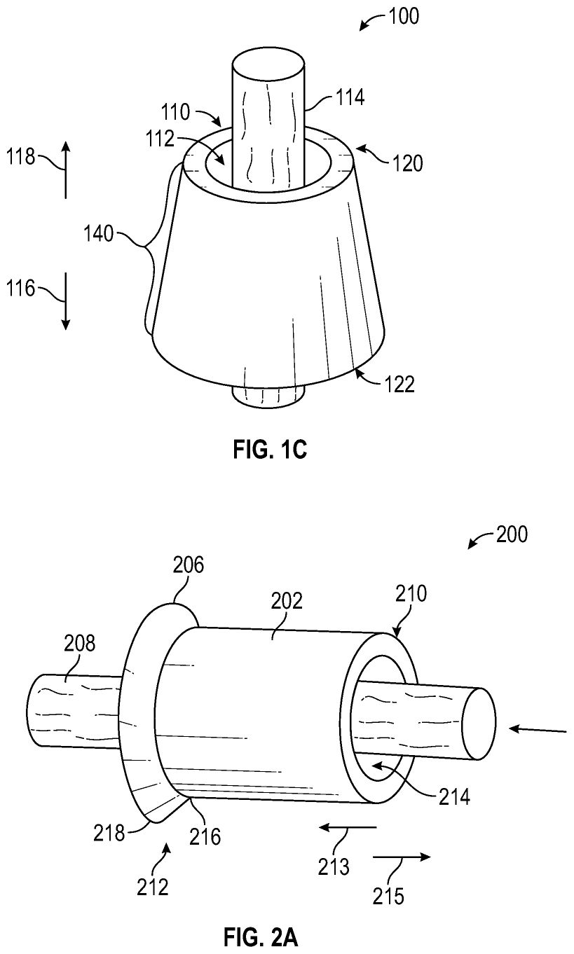

FIG. 1A illustrates an example of a fastener 100 according to one embodiment of the present disclosure. The fastener 100 may include a one-way tissue engagement feature 110 and an engagement feature 140. The one-way tissue engagement feature 110 may include an opening 112. The opening 112 is configured to receive a length of tether 114 passing through the opening 112. In certain embodiments, the tether 114 is a tissue such as a tissue graft. A tissue graft is one example of a tissue that can be used with embodiments of the disclosed solution.

The term “tether” is used herein to mean any strand or flexible member, natural or synthetic, able to join or connect or couple two structures or components. In one embodiment, a tether can join tissue of a patient and/or to be anchored in a bone tunnel or to hard tissue and useful in a surgical procedure. A tether may join two structures either directly by connecting directly to one structure or directly to the other or indirectly by connecting indirectly (by way of one or more intermediary structures) to one structure, to the other structure, or to both structures. In certain embodiments, “tether” refers to a flexible line or flexible member of natural material, natural biological material, biomaterial, biomimetic materials, manmade material, or a combination of these either in a single tether, a composite tether, or a plurality of tissue tethers that extend in parallel and/or may be woven or bonded together. In certain embodiments, a tether may be long and thin. In certain embodiments, a tether may be planar and/or may be elastic or inelastic (rigid). Examples of a tether include, but are not limited to, a thread, a string, a polymer thread or line, a tendon graft, a ligament graft, a hamstring graft, soft tissue, a tendon, a ligament, a suture, suture tape, a woven tether, a fibrous material, a cord, and/or any of these in combination with each other, and the like.

As used herein, a “graft,” “tissue graft,” and/or “bone graft” refers to a surgical procedure to move tissue (hard and/or soft tissue) from one site to another on the body, or from another creature, without bringing its own blood supply with the tissue. Instead, a new blood supply grows in after the tissue is placed. A similar technique where tissue is transferred with the blood supply intact is called a flap. (Search ‘Graft (surgery)’ on Wikipedia.com Apr. 21, 2021. Modified. Accessed Aug. 30, 2021.) “Graft” may also be used to refer to the tissue and/or synthetic composition used for a graft surgical procedure. Bone grafting is a surgical procedure that replaces missing bone in order to repair bone fractures. Bone generally has the ability to regenerate completely but may require a small fracture space and/or a scaffold to do so. Bone grafts may be autologous (bone harvested from the patient's own body, often from the iliac crest), allograft (cadaveric bone usually obtained from a bone bank), or synthetic (often made of hydroxyapatite (HA) or other naturally occurring and biocompatible substances) with similar mechanical properties to bone. Generally, bone grafts are expected to be reabsorbed and replaced as natural bone heals over a few months' time. (Search ‘Bone Grafting’ on Wikipedia.com Apr. 21, 2021. Modified. Accessed Aug. 30, 2021.) Certain grafts may include a combination of autograft, isograft, allograft, xenograft, and/or synthetic materials in a single bone graft composition. An example of such a compositions, include but is not limited to, Demineralized bone matrix (DBM). Bone graft compositions may include bone morphogenetic proteins (BMPs). As used herein, “allograft” refers to a type of tissue and/or organ graft in which the tissue or organ of the graft is from a donor of the same species but not the same genotype. The tissue may be soft tissue such as skin, ligament, tendon, fascia, fat muscle, fibrous tissue, blood vessels, lymph vessels, or nerves or hard tissue such as bone, tooth enamel, dentin, cementum, or cartilage. Bone grafts may be of an allograft type of a mixture of other graft types including allograft, autograft, and xenograft. Autograft refers to a type of tissue and/or organ graft in which the tissue or organ of the graft is from the patient. Xenograft refers to a type of tissue and/or organ graft in which the tissue or organ of the graft is from a donor of another species.

The opening 112 is large enough to accept an end the tether 114 passing through the opening 112. In the illustrated embodiment, the opening 112 has a circular cross-section shape. In certain embodiments, by analogy, the one-way tissue engagement feature functions with respect to the tether 114, much like a check valve functions with respect to a fluid. Like fluid flowing through a check valve, the tether 114 can only pass through the one-way tissue engagement feature in one direction.

FIG. 1B illustrates a side view of the fastener 100 of FIG. 1A. FIG. 1B illustrates that one portion (the ends of the tether 114 are not illustrated) of the tether 114 passes through the fastener 100 in a first direction 116. In one embodiment, the one-way tissue engagement feature 110 may be within the opening 112. The one-way tissue engagement feature 110 engages the length of tether 114 within the opening 112 such that the length of tether 114 can move through the opening 112 in the first direction 116 but not move through the opening 112 in a second direction 118 (or is at least restricted in movement through the opening 112 in the second direction 118). In this manner, the one-way tissue engagement feature 110 provides a one-way tissue engagement function, the tether 114 can only pass through the opening 112 in one direction. As used herein, “direction” refers to the line or course on which something or someone is moving or is aimed to move; or along which something is pointing or facing. (search “direction” on Merriam-Webster.com. Merriam-Webster, 2021. Web. 4 Aug. 2021. Modified.)

A pushing, pulling, or tension force applied to the tether 114 in the first direction 116 can/will cause the tether 114 to move through the one-way tissue engagement feature 110 in the first direction 116. Conversely, a pushing, pulling, or tension force applied to the tether 114 in the second direction 118 can/will not cause the tether 114 to move through the one-way tissue engagement feature 110 in the second direction 118. Instead, movement of the tether 114 in the second direction 118 will be stopped, retained, restricted, prevented, or mitigated by the one-way tissue engagement feature 110 of the fastener 100. In certain embodiments, the one-way tissue engagement feature 110 engages with the tether 114 such that a failure or tear of the tether 114 within the one-way tissue engagement feature 110 may be the only way to remove the one-way tissue engagement feature 110 from the tether 114.

Advantageously, the fastener 100 can be used in a variety of surgical procedures in which tether 114, suture, and/or one or more other flexible objects are to be fixed to other parts of the body, to each other, or to a separate implant. Such procedures include, but are not limited to, fixation of an autograft, allograft, xenograft, or artificial ligament to a bone such as a tibia, femur, humerus, radius, etc., fixation of a suture to a bone as in rotator cuff repair, tenodesis repair, fixation of a tissue graft within a bone tunnel at one or both ends, etc. Such flexible objects may be secured to various tissues, including hard tissues such as bone, flexible tissue such as cartilage, or even softer tissues.

A variety of mechanical configurations and designs may be used to implement the one-way tissue engagement feature 110. Each of these is considered within the scope of this disclosure. This disclosure includes exemplary embodiments that can serve as the one-way tissue engagement feature 110.

In one embodiment, the fastener 100 may include a distal end 120 and a proximal end 122. The distal end 120 may contact a body part of a patient as part of fixating a tether 114. For example, in a transosseous (through the bone) fixation procedure the distal end 120 may contact bone of a patient. The proximal end 122 may also contact a part of the body but not for purposes of fixation.

In one embodiment, the distal end 120, or a part of the distal end 120, such as a surface of the distal end 120, may serve as an engagement feature 140. The engagement feature 140 maintains contact between the fastener 100 and a part of the body. In certain embodiments, the engagement feature 140 maintains contact between the fastener 100 and a part of the body in response to tension applied to the tether 114 in the second direction 118 opposite the first direction 116. As the tension applied to the tether 114 in the second direction 118 increases the pressure that the engagement feature 140 applies to the part of the body in contact with the engagement feature 140 increases.

In one embodiment, the engagement feature 140 is configured to maintain contact between the fastener and a bone of the patient in response to tension applied to the tether in the second direction to keep the fastener in place relative to the bone. “Place” refers to a position or location for a component, structure, device, object, or person. In certain embodiments, place refers to a position, orientation, or location relative to one or more other objects or structures. In other embodiments, a place can refer to a geographic location.

As used herein, a “tension” refers to a force that is applied to both ends of a structure. The structure may have a variety of shapes including fat, thin, wide, elongated, or the like. In one example, a ligament such as a lateral collateral ligament may experience tension due to how the ligament is attached to a femur bone and tibia bone and stretched during flexing of the knee joint. In another example, a tether, suture, or other structure may experience and/or transfer tension as the structure is connected between an implant and an anchor, such as a bone anchor. As used herein, an “anchor” refers to an apparatus, instrument, structure, member, part, device, component, system, or assembly structured, organized, configured, designed, arranged, or engineered to secure, retain, stop, and/or hold, an object to or at a fixed point, position, or location. Often, an anchor is coupled and/or connected to a flexible member such as a tether, chain, rope, wire, thread, suture, suture tape, or other like object. Alternatively, or in addition, an anchor may also be coupled, connected, and/or joined to a rigid object or structure. In certain embodiments, an anchor can be a fixation device. Said another way, a fixation device can function as an anchor.

The engagement feature 140 connects to the one-way tissue engagement feature 110. Consequently, the one-way tissue engagement feature 110 secures the engagement feature 140 to the tether 114 and the engagement feature 140 can secure the tether 114 to a body part in contact with the engagement feature 140.

In one embodiment, the engagement feature 140 is a surface on the distal end 120 of a structure that includes the engagement feature 140. Alternatively, or in addition, the engagement feature 140 may be on the proximal end 122 of a structure that includes the engagement feature 140, or at any position between the distal end 120 and the proximal end 122.

FIG. 1C illustrates a side perspective view of an example of a fastener 100 according to one embodiment of the present disclosure. The fastener 100 includes the one-way tissue engagement feature 110, opening 112, and tether 114. In the illustrated embodiment, the fastener 100 may include a body that circumscribes the one-way tissue engagement feature 110.

In one embodiment, the engagement feature 140 extends from the body and faces at least partially toward the second direction 118 such that the engagement feature 140 contacts part of the bone of a patient. In the illustrated embodiment, the engagement feature 140 is embodied as an external surface of the fastener 100 that has a progressively increasing diameter as the external surface extends from the distal end 120 to the proximal end 122. Advantageously, this increased diameter can engage with tissue of a patient such as a bone surface or an internal wall of a bone tunnel of the patient. In this manner, the engagement feature 140 can engage with the tissue.

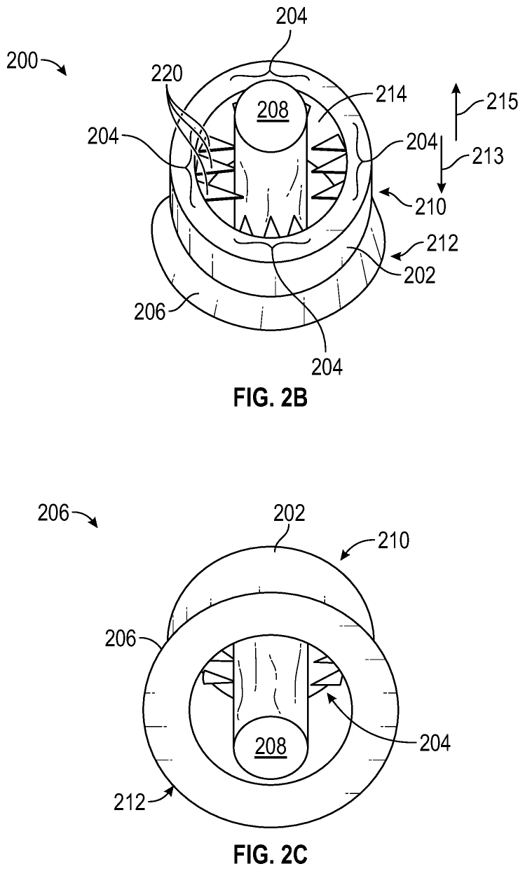

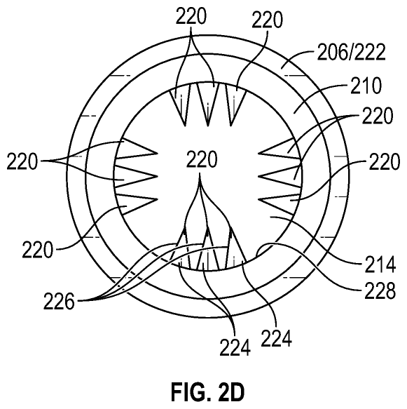

FIG. 2A illustrates a side perspective view of an example of a fastener 200 according to one embodiment of the present disclosure. The fastener 200 may include a body 202, a one-way tissue engagement feature 204, and an engagement feature 206. The fastener 200 secures a tissue 208 within the opening 214.

As explained herein, the tissue 208 may be a manmade tissue or a biological tissue and/or a combination of these. For example, in one embodiment, the tissue 208 is a hamstring graft (i.e., Hamstring tendon autograft (GST)) that includes one or more sutures and/or suture tape secured to one end or extending along the length of the graft. In one embodiment, suture(s) or suture tape may extend along the length of the tissue 208 to add structural strength to the graft and may enhance the engagement of the one-way tissue engagement feature 204 of the tissue 208.

The body 202 has a distal end 210 and a proximal end 212. The body 202 provides structural support for the fastener 200. The body 202 may have a variety of shapes and sizes. In one embodiment, the body 202 is an elongated body. The body 202 may have a circular longitudinal cross section. The body 202 may be tube shaped. “Tube” refers to any structure or component that is hollow and cylindrical in shape. (Search “tube” on wordhippo.com. WordHippo, 2022. Web. Accessed 21 Jul. 2022. Modified.) In the illustrated embodiment, the body 202 circumscribes the one-way tissue engagement feature 204.

In certain embodiments, the distal end 210 and proximal end 212 serve as the distal end 210 and proximal end 212 for the fastener 200. The opening 214 extends between the distal end 210 and the proximal end 212. In the illustrated embodiment, an engagement feature 206 is proximal to the proximal end 212 and a first direction (e.g., one direction 213) starts at the distal end 210 and continues to the proximal end 212.

The one-way tissue engagement feature 204 is connected to the body 202. The one-way tissue engagement feature 204 may include an opening 214 that extends through the body 202. The one-way tissue engagement feature 204 serves to secure the tissue 208 such that the tissue 208 can only move with respect to the fastener 200 in one direction 213. Specifically, the one-way tissue engagement feature 204 may secure the tissue 208 within the opening 214 such that the tissue 208 can move in one direction (longitudinally in the illustrated embodiment) within the opening 214 but not the opposite direction 215.

The engagement feature 206 is configured to contact and/or engage bone, an implant, or other tissue, in response to threading, pulling, or pushing the tissue 208 through the one-way tissue engagement feature 204. In the illustrated embodiment, the engagement feature 206 may be a flange that circumscribes the body 202. The flange may be a separate part connected to the body 202. Alternatively, or in addition, the flange may be formed as part of and be integrated with the body 202. The engagement feature 206 may include a distal end 216 and a proximal end 218.

In certain embodiments, the engagement feature 206 may be referred to as a stop. The body 202 may have a cylindrical shape and the stop 206 may connect to the cylindrical body 202 at or near the proximal end 212. As used herein, a “stop” refers to an apparatus, instrument, structure, member, device, component, system, or assembly structured, organized, configured, designed, arranged, or engineered to prevent, limit, impede, stop, or restrict motion or movement and/or operation of the another object, member, structure, component, part, apparatus, system, or assembly. Alternatively, or in addition, the one-way tissue engagement feature 204 may be referred to as a stop.

In one embodiment, the engagement feature 206 or stop 206 may include a lip. “Lip” refers to a structure that extends and/or projects from another structure. In certain embodiments, a lip has a form and look of a lip on a face of a human. Often, a lip is a structure found at or near an opening such as a mouth, tube, or tunnel. The lip may circumscribe the cylindrical body 202. The lip can be sized to contact a surface of bone around a bone tunnel that accepts the fastener 200.

FIG. 2B is a perspective view of the fastener of FIG. 2A from the distal end 210, according to one embodiment. In the illustrated embodiment, the engagement feature 206 is near the proximal end 212 of the fastener 200. The one-way tissue engagement feature 204 may include one or more teeth 220 that extend into the opening 214. In certain embodiments, a variety of other, additional, or alternative protrusions, bias members, barbs, bumps, arms, or other interference structures may be used in place of, or together with, the one or more teeth 220.

As used herein, “tooth” or “teeth” refers to a structure(s) including a natural body part of a person or animal and/or a structure that functions and/or is structured like a similar structure of a person or animal. A tooth can include a base, one or more sides, and an edge. The edge may come to a point or may taper to a sharp edge. The edge or point may serve to tear or cut through or otherwise engage other objects such as food or other materials. A tooth can also refer to a projection resembling or suggesting the tooth of an animal or person in shape, arrangement, or action. (search “tooth” on Merriam-Webster.com. Merriam-Webster, 2021. Web. 4 Aug. 2021. Modified.) A tooth may be made of natural materials such as bone-like material and/or enamel or may be made of a variety of materials including, but not limited to, metal, plastic, ceramic, wood, fiberglass, acrylic, carbon, biocompatible materials, biodegradable materials or the like.

The teeth 220 are configured to engage a tether (and/or tissue 208) within the opening 214 such that the tether can move through the opening 214 in the first direction 213 and not move through the opening in the second direction 215. The one or more teeth 220 are connected to the body 202 and are configured, arranged, and/or positioned such that the one or more teeth 220 permit the tissue 208 to slide by the one or more teeth 220 when the tissue 208 moves in first direction 213 and engage and prevent, limit, or interfere with movement of the tissue 208 past the one or more teeth 220 when the tissue 208 is pushed, pulled, or biased in the second direction 215. Thus, the one or more teeth 220 engage the tissue 208 within the opening 214 such that the tissue 208 can move through the opening 214 in the first direction 213 and not move through the opening 214 in the second direction 215 opposite the first direction 213.

As the tissue 208 is pushed, pulled, placed under tension, or biased in the second direction 215, the one or more teeth 220 may pierce/bite into the tissue 208. The piercing or biting of the one or more teeth 220 may cause the one or more teeth 220 to go into the tissue 208 a relatively small distance initially. But, as the tension increases or more force is applied to move the tissue 208 in the second direction 215, the one or more teeth 220 may sink or seat deeper into the tissue 208 which enhances the engagement and further secures the tissue 208 to prevent the tissue 208 from moving in the second direction 215. In this manner, the one or more teeth 220 may enable the one-way tissue engagement feature 204 to permit the tissue 208 to move in one direction, but not in an opposite direction.

In the illustrated embodiment, the fastener 200 includes four sets of one or more teeth 220: a top set, a bottom set, and two side sets. Of course, the number of teeth 220, their position within the opening 214, the configuration of the one or more teeth 220 relative to each other can be different in different embodiments of this disclosed technology.

FIG. 2B illustrates an embodiment of the fastener 200 which may be referred to as a one-way tether lock for anchoring a tissue graft. “One-way tether lock” refers to a structure, component, apparatus, or device configured, designed, and/or engineered to securely engage a tether. Specifically, a one-way tether lock is configured to engage with a tether that extends into the one-way tether lock in a first direction and hinder, mitigate, arrest, or stop movement of the tether or one-way tether lock relative to each other in a second direction that is not the first direction. A tissue graft is one example of a tether or tissue that can be used with different embodiment of the present disclosure.

The one-way tether lock 200 includes a cylindrical body 202 having a proximal end 212, a distal end 210, and an opening 214 that extends from the distal end 210 to the proximal end 212. The one-way tether lock 200 also includes a set of teeth 220 that extend into the opening 214. The set of teeth 220 are configured to permit passage of a tissue graft from the distal end 210 toward the proximal end 212 and constrain passage of the tissue graft from the proximal end 212 toward the distal end 210.

FIG. 2C is a perspective view of the fastener of FIG. 2A from the proximal end 212, according to one embodiment. In the illustrated embodiment, the one-way tissue engagement feature 204 may comprise a single row of one or more teeth 220. Of course, multiple rows of one or more teeth 220 may be used in certain embodiments.

FIG. 2D is an end view of the fastener 200 of FIG. 2A from the distal end 210, according to one embodiment. In one embodiment, the engagement feature 206 is a stop 222 connected to the body 202 and configured to engage with a bone of a patient. The stop 222 may be configured to prevent the fastener 200 from moving into a bone tunnel, such as a femoral tunnel or tibial tunnel, in response to tension from a tissue 208 within the opening 214. The stop 222 may be a flange that circumscribes the body 202.

In one embodiment, the one or more teeth 220 may have a broad base 224 that tapers to a tip 226 (also referred to as a point) to facilitate engagement with the tissue 208. “Tip” refers to an end of a structure. Often, a tip includes tapered edges that come together to form and edge and/or a point. A tip can be sharp or blunt. A tip can have a variety of shapes, sizes, and cross sections. A tip can have a circular cross section. The base 224 can connect with an interior wall 228. As used herein, a “base” refers to a main or central structure, component, or part of a structure. A base is often a structure, component, or part upon which, or from which other structures extend into, out of, away from, are coupled to, or connect to. A base may have a variety of geometric shapes and configurations. A base may be rigid or pliable. A base may be solid or hollow. A base can have any number of sides. In one embodiment, a base may include a housing, frame, or framework for a larger system, component, structure, or device. In certain embodiments, a base can be a part at the bottom or underneath a structure designed to extend vertically when the structure is in a desired configuration or position.

The one or more teeth 220 may have a variety of sizes and shapes, including different sizes between them. The one or more teeth 220 extend from the interior wall 228 of the opening 214. “Wall” refers to a structure that serves to separate one part or area of a structure from another. A wall may also serve as a barrier that prevents passage of fluid or solids between two parts or areas of a device or structure. A wall can also serve to define a boundary between one or more sections of a structure or device. For example, a wall can define an interior chamber or room and/or an exterior chamber, area, or room.

Referring to FIGS. 2B and 2D, one or more teeth of the one or more teeth 220 may extend perpendicular to the wall. Alternatively, or in addition, one or more teeth of the one or more teeth 220 may extend from the wall at an angle. For example, in one embodiment, the one or more teeth 220 may extend from the wall and point towards the proximal end 212 at an angle between 0 and 85 degrees. Said another way, each tooth of the teeth 220 may be oriented such that the tip 226 is displaced in a first direction (e.g., one direction 213 in FIG. 2A).

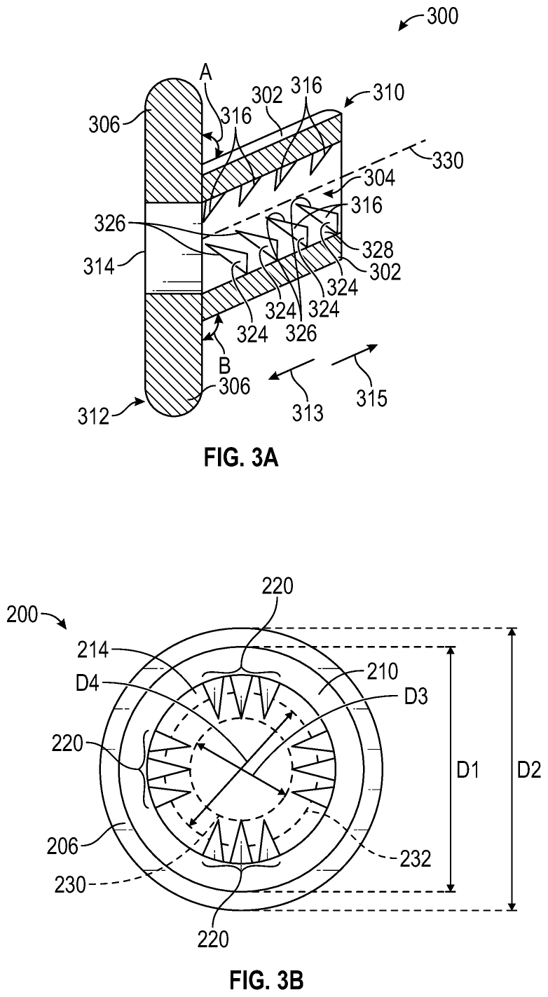

FIG. 3A is a cross-sectional view of a fastener 300 taken along a longitudinal axis of the fastener, according to one embodiment. The fastener 300 may include a body 302, a one-way tissue engagement feature 304, and a bone engagement feature 306. For clarity, a tissue is not shown in FIG. 3. The fastener 300 may include a distal end 310, a proximal end 312, and an opening 314. The bone engagement feature 306 may include one or more teeth 316. Each tooth 316 may include a base 324 and a tip 326. The longitudinal axis 330 is parallel to a first direction 313 (one direction) and a second direction 315 (opposite direction).

In the illustrated embodiment, the fastener 300 includes teeth 316 wherein each tooth 316 is connected to an interior wall 328 of the body 302 at the base 324. The teeth 316 may extend from the wall 328 at various angles, including acute, obtuse, and perpendicular. In certain embodiments, the teeth 316 include a tip 326 (or an edge) that is oriented in the first direction 313, towards the proximal end 312. Said another way, the tip 326 is displaced in the first direction 313 relative to the base 324.

FIG. 3A illustrates that the tip 326 of the teeth 316 can include a tapered edge, and/or come to a point. Tapered edge can facilitate engagement with a tether in the opening 314. As used herein, “edge” refers to a structure, boundary, or line where an object, surface, or area begins or ends. An edge can also refer to a boundary or perimeter between two structures, objects, or surfaces. An edge can also refer to a narrow part adjacent to a border. (search “edge” on Merriam-Webster.com. Merriam-Webster, 2021. Web. 3 Aug. 2021. Modified.) In certain embodiments, an edge can be a one dimensional or a two dimensional structure that joins two adjacent structures or surfaces. Furthermore, an edge may be at a perimeter of an object or within a perimeter or boundary of an object.

Advantageously, the tapered edge tip 326 and orientation of the teeth 316 can facilitate passing a tether through the opening 314 in the first direction 313. Because of the orientation of the teeth 316 the tether slides past the teeth 316 as the tether moves in the first direction 313. The tapered edge of the teeth 316 engage with, and/or extend into, the tether as the tether moves or is pulled or pushed in the second direction 315, for example due to a tension force in the second direction 315.

In certain embodiments, the teeth 316 are configured to function while remaining within the perimeter of the opening 314. FIG. 3A illustrates that the length of the teeth 316 and/or the configuration of the teeth 316 are such that the tip 326 of at least one tooth of the set of teeth 316 remains within the opening 314. Alternatively, or in addition, each of the teeth 316 are configured such that the tips 326 remain within the opening 314. In other words, the tips 326 can be configured to not extend outside the opening 314 beyond the proximal end 312. This can be advantageous because the tips 326 would not contact or rub against other patient tissue such as skin and thus would not cause irritation or discomfort.

The fastener 300 is similar to the fastener 100 and the fastener 200. But, the fastener 300 differs from the fastener 100 and fastener 200 because the bone engagement feature 306 connects to the body 302 at an angle that is not perpendicular. The bone engagement feature 306 may connect to the body 302 at a non-perpendicular angle in order to accommodate a bone tunnel that extends into a bone from a surface at an angle that is not perpendicular to the surface.

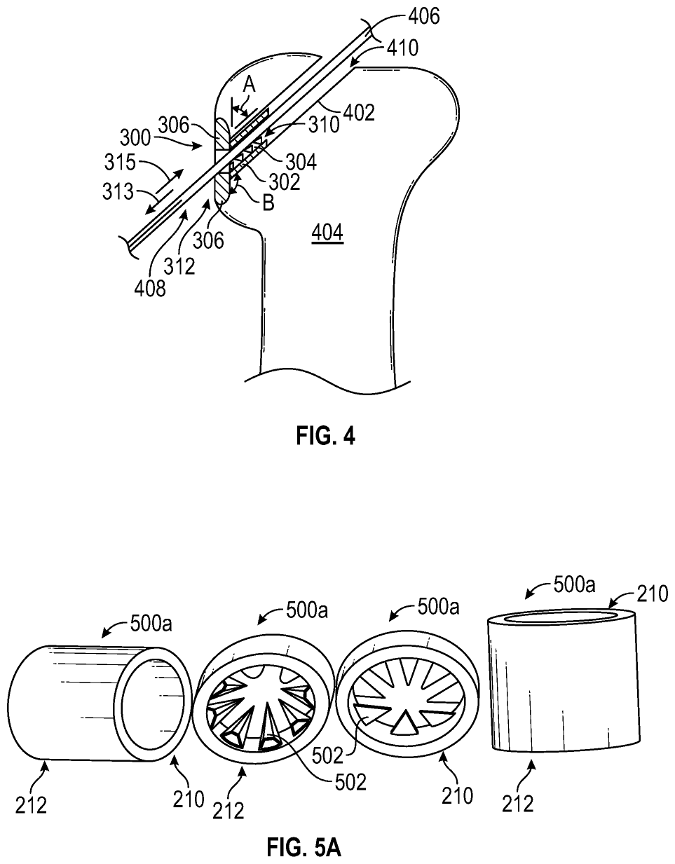

FIG. 3A illustrates an embodiment in which the bone engagement feature 306 connects to the body 302 at an angle A that is not perpendicular, is not 90 degrees. Similarly on a side of the body 302 opposite angle A, the bone engagement feature 306 may connect to the body 302 at an angle B that is also not perpendicular and may be corresponding to angle A. For example, if angle A is 65 degrees, angle B may be a complementary angle such as 115 degrees. In certain embodiments, the bone engagement feature 306 may connect to the body 302 at an angle that substantially matches an angle of a bone tunnel relative to a cortical surface of the bone. (See FIG. 4 for an example). “Cortical bone” refers to a type of bone tissue. Cortical bone is a type of bone tissue typically found between an external surface of a bone and an interior area of the bone. Cortical bone is more dense and typically stronger structurally than other types of bone tissue.

Thus, the bone tunnel may extend from a surface of a bone at first angle not perpendicular to the surface (e.g., Angle A). The bone engagement feature 306 may be embodied as a lip 306 that extends from the cylindrical body 302 at a second angle (e.g., Angle B) that corresponds to the first angle. A diameter of the bone tunnel may be larger than a diameter of the cylindrical body 302 and smaller than a diameter of the lip 306. The angles of the bone engagement feature 306 relative to the body 302 and in relation to the angle of a bone tunnel can facilitate the engagement between the bone engagement feature 306 and a surface of the bone around the bone tunnel.

FIG. 3B is an end view from a distal end of a fastener, such as fastener 200, according to one embodiment. FIG. 3B illustrates different diameters of the fastener 200. The fastener 200 includes a body diameter D1 that can be sized to fit within a bone tunnel. In one example, body diameter D1 may be smaller than a diameter of the bone tunnel. The bone tunnel may be configured to accept the fastener 200. In one embodiment, the fastener 200 also includes an engagement feature 206 having a diameter D2 that is greater than the body diameter D1. In certain embodiments, the engagement feature's diameter D2 can be sized to serve as a stop and may be part of a cylindrical body 202 of the fastener 200.

In another embodiment, the bone tunnel may include a tapering diameter (tapering from a first size near an opening of the tunnel down smaller to a second size further into the tunnel from the opening) and the engagement feature 206 may be integrated with the body 202 and may have a diameter D2 that substantially matches D1.

In certain embodiments, the teeth 220 are pliable and configured to bend in the one direction 213/313, a first direction (towards the proximal end 212/312). In particular, the teeth 220 can bend toward the proximal end 212/312 as a tether is moved through the opening 214 in the first direction, one direction 213/313. In the illustrated embodiment, the teeth 220 define an initial diameter passage 230 in the opening 214 having a diameter D3. “Initial diameter passage” refers to a passage that starts with a first diameter. In certain embodiments, the structure or structures that define the initial diameter passage are configured to enable the diameter to change from an initial diameter passage to one or more of a larger diameter and a smaller diameter. As used herein, “passage” refers to a duct, a vessel, an opening, a void, or other channel in a body of a an apparatus, instrument, structure, member, device, component, system, or assembly. In certain embodiments, a passage is narrow and longer than the passage is wide. (Search “passage” on wordhippo.com. WordHippo, 2021. Web. Accessed 15 Nov. 2021. Modified.)

Because the teeth can bend towards the proximal end 212, the bending teeth 220 can also define a second diameter passage 232 having a diameter D4 greater than the diameter D3 of the initial diameter passage 230. Advantageously, the ability of the teeth 220 to define different diameter passages 230/232 facilitates the use and operation of the fastener 200. The fastener 200 can naturally accept tethers or tissue of various and/or variable diameters and still perform the desired one-way fastening and securement.

FIG. 4 is a cross-sectional view of a fastener 300 taken along a longitudinal axis 330 (See FIG. 3A) of the fastener 300 positioned within a bone tunnel 402 of a bone 404, according to one embodiment. As used herein, “tunnel” refers to a duct, an opening, a void, a passage, or other channel in a body of an apparatus, instrument, structure, part, bone, member, device, component, system, or assembly. In certain embodiments, a tunnel is narrower and longer than the tunnel is wide. A tunnel can have a variety of shapes and/or cross-sections and may be straight or include curves or bends. In certain embodiments, a tunnel may have a circular cross section.

The fastener 300 may include a body 302, a one-way tissue engagement feature 304, and a bone engagement feature 306. The fastener 300 secures a tissue 406 and has a distal end 310 and a proximal end 312. The fastener 400 is illustrated in a deployed state such that part of the body 302 extends into the bone tunnel 402 and secures the tissue 406. The bone tunnel 402 includes a proximal end 408 and a distal end 410.

In one embodiment, the fastener 300 is deployed within the bone tunnel 402. The bone tunnel 402 may be formed within a bone 404 (e.g., a proximal end of a tibia) as part of an anterior cruciate ligament (ACL) replacement procedure. During such a procedure, a distal end of the tissue 406 may be fixed to a femur (not shown) using either embodiments of the disclosed solution or existing fasteners and/or other techniques. With the distal end of the tissue 406 secured, a proximal end of the tissue 406 may be threaded through the opening 314 of the fastener 300. The tissue 406 may be pulled through the opening 314 such that the one-way tissue engagement feature 304 engages the tissue 406. Next, a user (e.g. surgeon) can pull the tissue 406 through the fastener 300 to tension the graft within the bone tunnel 402.

Once the one-way tissue engagement feature 304 engages the tissue 406 the user has full control of how much tension is placed within the graft (e.g., tissue 406). Advantageously, as the tissue 406 pass through the fastener 300, the fastener 300 advances along the tissue 406 (e.g., graft) and seats within the bone tunnel 402. This process of moving the fastener 300 along the tissue 406 may be referred to herein as synching of the fastener 300. During the steps of synching of the fastener 300, a surgeon may advantageously check, monitor, and manage tension in the graft with a knee in different positions of flexion. Once sufficient checks of tension are done, at one or more flexion positions of a knee, the user may further tighten or synch the fastener 300. Advantageously, the fastener 300 secures the tissue 406 with a sufficient force within the bone tunnel 402 to enable the patient to return to regular activities quickly. In one embodiment, the fastener 300 secures the tissue 406 to retain the tissue 406, such as a graft, when graft experiences 150-500 Newtons (N) of force due to activities of daily living and rehabilitation exercises.

In certain embodiments, the body 302 may be sized to readily fit within the bone tunnel 402 and the bone engagement feature 306 may abut the cortex of the bone 404 around the bone tunnel 402. In another embodiment, the bone tunnel 402 may be configured to accept the bone engagement feature 306 within at least part of the bone tunnel 402. For example, a surgeon may resect an end of the bone tunnel 402 with a larger diameter than the remainder of the bone tunnel 402 such that the bone engagement feature 306 becomes countersunk and thus flush with a cortex of the bone 404.

Those of skill in the art will recognize that the bone engagement feature 306 may be embodied in a variety of configurations. For example, rather than a flange that circumscribes the body 302, the bone engagement feature 306 may comprise one or more arms that extend from the body 302 and may be positioned to engage with the cortex or a countersink depression in the bone 404. Alternatively, or in addition, the bone engagement feature 306 may include a stop configured to constrain movement of the fastener 300 from a proximal end 408 of the bone tunnel 402 toward a distal end 410 of the bone tunnel 402. In one embodiment, the stop may be a lip (e.g. bone engagement feature 306) formed near a proximal end 312 of the fastener 300. The lip may circumscribe the body 302 (which may be cylindrical, in one embodiment). The lip may be sized to contact a surface of the bone 404 around the bone tunnel 402 that accepts the fastener 300.

In certain embodiments, the body 302 and/or bone engagement feature 306 may be contoured and/or shaped to have a minimal profile once deployed in the bone tunnel 402. For example, based on an angle of entry for the bone tunnel 402 the parts of the fastener 300 outside the bone tunnel 402 may be shaped and/or contoured to match a contour of a patient's body where the fastener 300 is deployed. FIG. 4 also illustrates that an angle A between the bone engagement feature 306 and the body 302 may be designed, or fabricated, such that the bone engagement feature 306 maintains a low profile when installed. In certain embodiments, the angle may be between 90 degrees and 15 degrees.

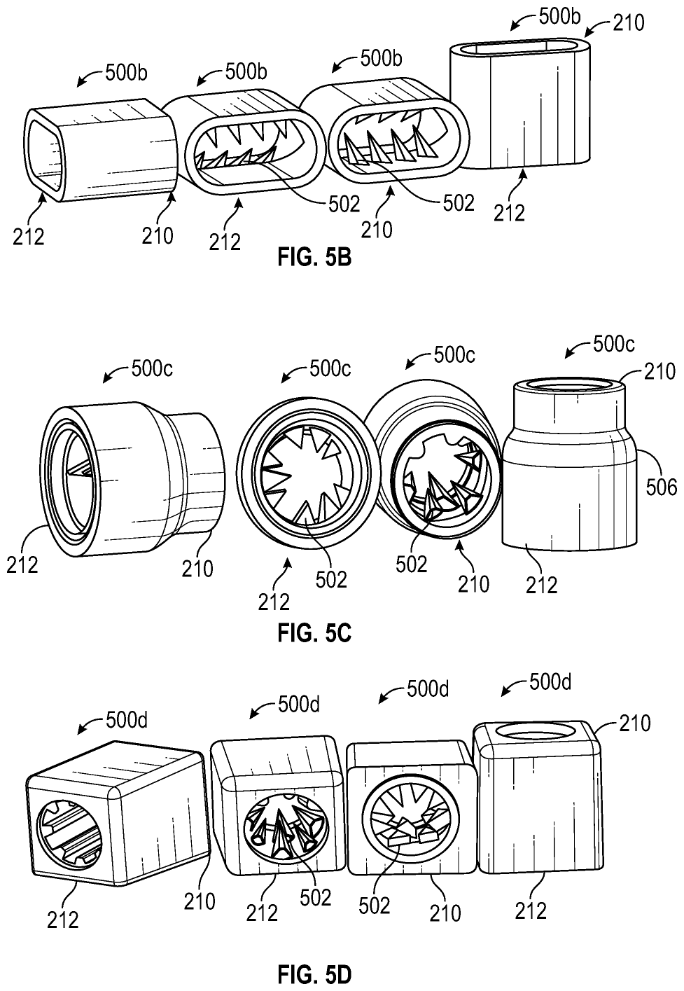

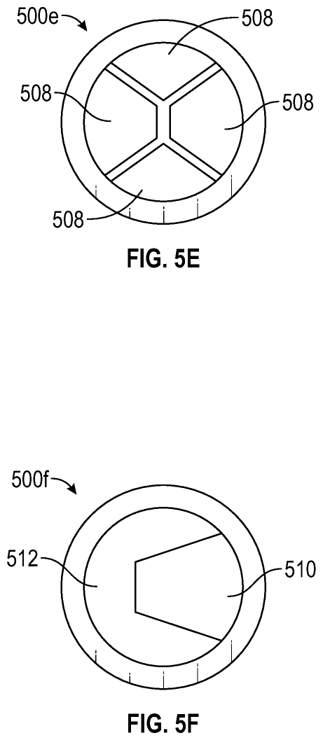

FIG. 5A is a side perspective view, end perspective view, opposite end perspective view, and a side perspective view of a fastener 500a having a circular longitudinal cross-section in four different orientations, according to one embodiment. In the illustrated embodiment, the teeth 502 are shaped to come to a point. From left to right, the first orientation is with the fastener 500a on its side. The next orientation shows the fastener 500a from the proximal end 212. The next orientation shows the fastener 500a from the distal end 210. The next orientation shows the fastener 500a standing on end.

FIG. 5B is a side perspective view, end perspective view, opposite end perspective view, and a side perspective view of a fastener 500b having an elliptical longitudinal cross-section in four different orientations, according to one embodiment. In the illustrated embodiment, the teeth 502 are shaped to come to a point. From left to right, the first orientation is with the fastener 500b on its side. The next orientation shows the fastener 500b from the proximal end 212. The next orientation shows the fastener 500b from the distal end 210. The next orientation shows the fastener 500b standing on end.

FIG. 5C is a side perspective view, end perspective view, opposite end perspective view, and a side perspective view of a fastener 500c having a circular longitudinal cross-section in four different orientations, according to one embodiment. In the illustrated embodiment, the teeth 502 are shaped to come to a point. From left to right, the first orientation is with the fastener 500c on its side. The next orientation shows the fastener 500c from the proximal end 212. The next orientation shows the fastener 500c from the distal end 210. The next orientation shows the fastener 500c standing on end.

The embodiment of FIG. 5C includes an engagement feature 506 integrated into the body of the fastener 500c. In certain embodiments, the engagement feature 506 is a shoulder formed in a wall of the body and circumscribing the opening. The shoulder may be sized to engage the cortex of a bone to fix a tissue and the fastener 500c to the bone.

FIG. 5D is a side perspective view, end perspective view, opposite end perspective view, and a side perspective view of a fastener 500d having a square longitudinal cross-section in four different orientations, according to one embodiment. Or course, other polygonal longitudinal cross-sections can be used to implement embodiments of the solution in this disclosure. In the illustrated embodiment, the teeth 502 are shaped to come to a point. From left to right, the first orientation is with the fastener 500d on its side. The next orientation shows the fastener 500d from the proximal end 212. The next orientation shows the fastener 500d from the distal end 210. The next orientation shows the fastener 500d standing on end.

FIG. 5E is an end view of a one-way tissue engagement feature, according to one embodiment. FIG. 5E illustrates a different type of one-way tissue engagement feature. The one-way tissue engagement feature may include four teeth 508 that extend from a wall of an opening in the fastener 500e. The teeth 508 may be align longitudinally within the opening or may be offset with respect to each other. The teeth 508 have be shaped as a polygon with a broad base and a flat top that does not come to a point.

FIG. 5F is an end view of a one-way tissue engagement feature, according to one embodiment. FIG. 5F illustrates a different type of one-way tissue engagement feature. The one-way tissue engagement feature may include a single tooth 510 that extends from a wall of an opening 512 in the fastener 500f. The single tooth 510 may extend beyond a radius of the opening 512. The single tooth 510 may be shaped as a polygon with a broad base and a flat top that does not come to a point. Those of skill in the art will appreciate that the one-way tissue engagement feature may be implemented in a variety of ways and that this disclosure includes just a few examples of those implementations, each of which is considered within the scope of this disclosure as claimed.

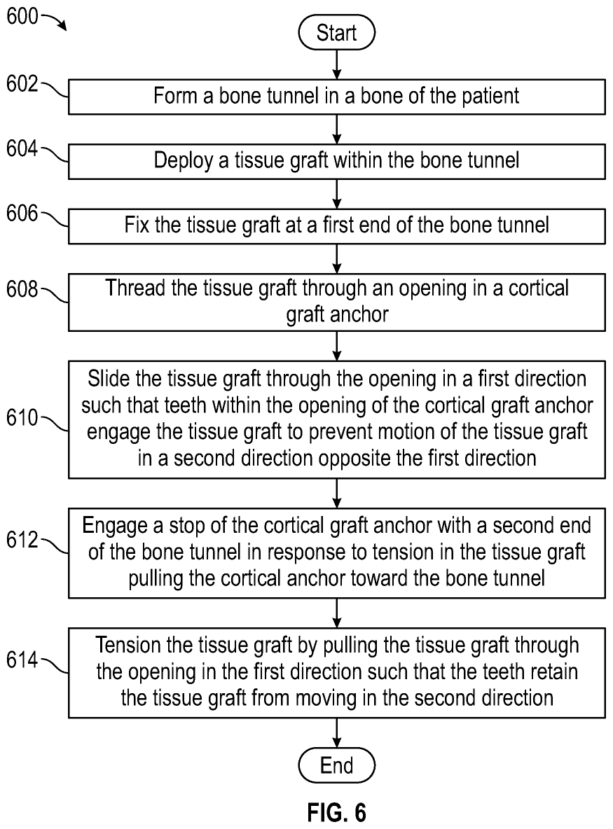

FIG. 6 illustrates one example of a method 600 for securing tissue or a tether within, or to, a body part of a patient, according to one embodiment. Referring to FIGS. 4 and 6, the method 600 starts with a user, such as a surgeon, forming 602 a bone tunnel 402 within a bone of a patient. Conventional tools and techniques may be used to form the bone tunnel 402. Next, the surgeon deploys 604 a tissue graft within the bone tunnel 402. A tissue graft is one example of a tissue that can be used with embodiments of the disclosed solution. Conventional tools and techniques may be used to deploy tissue graft within the bone tunnel 402.

Next, the surgeon fixes 606 the tissue graft at a first end of the bone tunnel 402 (e.g., at a distal end 410 of the bone tunnel 402). Conventional tools and techniques may be used to deploy tissue graft within the bone tunnel 402. Alternatively, or in addition, a fastener (e.g., fastener 100, fastener 200, fastener 300) according to the embodiments disclosed herein may be used to fix 606 the tissue graft at the first end of the bone tunnel 402 (e.g., the distal end 410). Next, the surgeon threads 608 the tissue graft through an opening in a cortical graft anchor (e.g., fastener 300). For example, in one embodiment, a surgeon may pass a free end of the tissue through the opening 314 starting from the distal end 310 and threading 608 the tissue 406 through until the free end exits the cortical graft anchor at the proximal end 312. “Cortical graft anchor” refers to an anchor that secures a graft. Often, the graft is a soft tissue graft. In certain embodiments, a cortical graft anchor is designed, engineered, and/or configured for securing the graft to cortical bone. However, a cortical graft anchor is not limited to securing the graft only to cortical sections of bone. In certain embodiments, a cortical graft anchor can secure a graft to other body parts or other parts of bone, such as cancellous parts of bone.

Next, the surgeon slides 610 the tissue graft through the opening 314 in a first direction 313 such that one or more teeth 316 within the opening 314 engage the tissue graft to prevent motion of the tissue graft, in a second direction 315 opposite the first direction 313.

Next, in one embodiment, a surgeon may engage 612 a stop of the cortical graft anchor (e.g., bone engagement feature 306 of fastener 300) with bone (e.g., the cortex) at a second end (e.g., the proximal end 408) of the bone tunnel 402. Specifically, the bone engagement feature 306 may engage with the bone 404 in response to tension in the tissue graft pulling the cortical graft anchor toward the bone tunnel 402.

Next in certain embodiments, the surgeon may tension 614 the tissue graft by pulling the tissue graft (e.g., tissue 406) through the opening 312 in the first direction 313 (e.g., toward the proximal end 312 of the fastener 300) such that one or more teeth 316 retain the tissue graft from moving in the second direction 315. Tension placed in the tissue graft by the surgeon and/or movement, such as flexion of a joint, may pull the tissue graft towards a distal end 310 of the fastener 300. However, the one or more teeth 316 bite and engage with the tissue graft to prevent movement of the tissue graft in the second direction 315. Then, the method 600 ends.

In one embodiment, the method 600 may include a step of forming a cutout around a second end (e.g., a proximal end 312) of the bone tunnel 402. “Cutout” refers to a hole or space created when something is removed, such as by cutting, or is not formed in the space of the cutout. Cutout may also refer to a piece of material cut out of something. (Search “cut-out” on wordhippo.com. WordHippo, 2022. Web. Accessed 21 Jul. 2022. Modified.) The cutout may be sized to accept a stop (e.g., a bone engagement feature 306) of the cortical graft anchor. Next, a surgeon may seat the stop with the cutout at the second end (e.g., a proximal end 312) of the bone tunnel 402. The stop may be seated as a user slides 610 the tissue graft and/or as a user tensions 614 the tissue graft. The cutout can facilitate counter sinking the fastener below the surface of the bone 402 around the bone tunnel 402.

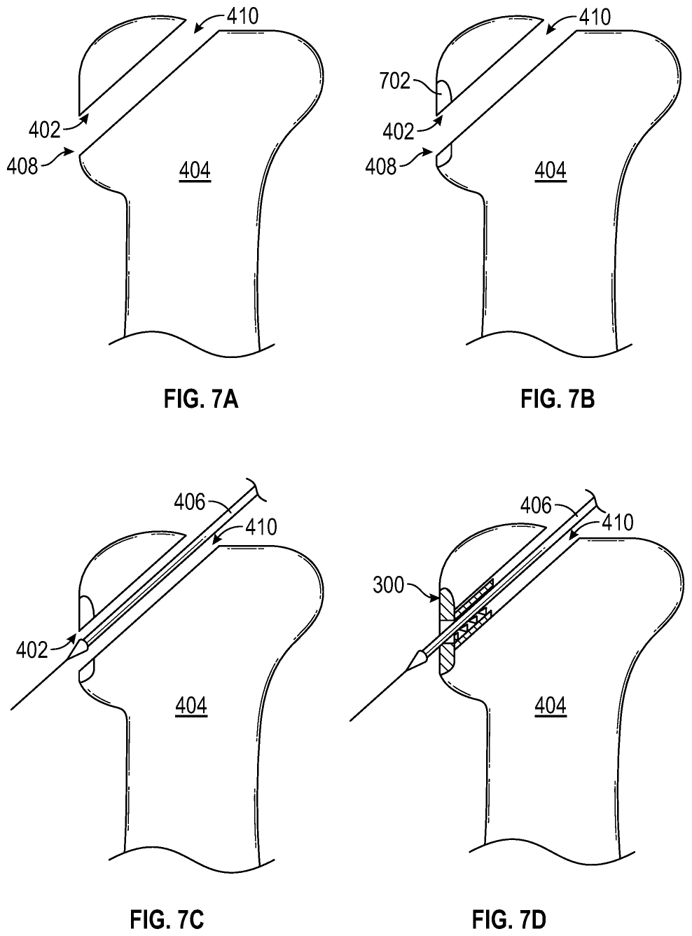

FIGS. 7A-7D illustrate cross-sectional views of different stages of practicing a method according to one embodiment of the present disclosure. FIG. 7A illustrates a proximal end of a bone 404 after a surgeon has formed 602 a bone tunnel 402 in the bone 404. FIG. 7B illustrates the bone tunnel 402 after a surgeon has formed 602 a cutout 702 at the proximal end 408 of the bone tunnel 402 in the bone 404. FIG. 7C illustrates the bone tunnel 402 with a tissue 406 deployed within the bone tunnel 402. A free end of the tissue 406 extends beyond the proximal end 408 of the bone tunnel 402. FIG. 7D illustrates the bone tunnel 402 after a surgeon has deployed the a cortical graft anchor (e.g., fastener 300) at the proximal end 408 of the bone tunnel 402 in the bone 404. The tissue 406 may already be under tension or a surgeon may next pull the tissue 406 toward the proximal end 408 of the bone tunnel 402 to add tension to the tissue 406.

Advantageously, the present disclosure provides a fastener that can be readily deployed and removed if a revision procedure is performed. For example, the fastener can be made from a variety of materials including, but not limited to, metal, plastic, ceramic, wood, fiberglass, acrylic, carbon, biocompatible materials, biodegradable materials or the like. For a revision procedure, a surgeon may cut the fastener longitudinally into two pieces such that the one-way tissue engagement feature can be disengaged from the tissue and the revision performed.

As used herein, a “fixation” or “fixation device” refers to an apparatus, instrument, structure, device, component, member, system, assembly, step, process, or module structured, organized, configured, designed, arranged, or engineered to connect two structures either permanently or temporarily. The two structures may be one or the other or both of manmade and/or biological tissues, hard tissues such as bones, teeth or the like, soft tissues such as ligament, cartilage, tendon, or the like. In certain embodiments, fixation is used as an adjective to describe a device or component or step in securing two structures such that the structures remain connected to each other in a desired position and/or orientation. Fixation devices can also serve to maintain a desired level of tension, compression, or redistribute load and stresses experienced by the two structures and can serve to reduce relative motion of one part relative to others. Examples of fixation devices are many and include both those for external fixation as well as those for internal fixation and include, but are not limited to pins, wires, Kirschner wires (K-wires), screws, anchors, bone anchors, plates, bone plates, intramedullary nails or rods or pins, implants, interbody cages, fusion cages, and the like.

As used herein, a “fixator” refers to an apparatus, instrument, structure, device, component, member, system, assembly, or module structured, organized, configured, designed, arranged, or engineered to connect two bones or bone fragments or a single bone or bone fragment and another fixator to position and retain the bone or bone fragments in a desired position and/or orientation. Fixators can also serve to redistribute load and stresses experienced by bone(s) and/or body parts and can serve to reduce relative motion of one part relative to others. Examples of fixators include both those for external fixation as well as those for internal fixation and include, but are not limited to pins, wires, Kirschner wires, screws, anchors, bone anchors, plates, bone plates, intramedullary nails or rods or pins, implants, interbody cages, fusion cages, and the like.

As used herein, an “opening” refers to a gap, a hole, an aperture, a space or recess in a structure, a void in a structure, or the like. In certain embodiments, an opening can refer to a structure configured specifically for receiving something and/or for allowing access. In certain embodiments, an opening can pass through a structure. In other embodiments, an opening can exist within a structure but not pass through the structure. An opening can be two-dimensional or three-dimensional and can have a variety of geometric shapes and/or cross-sectional shapes, including, but not limited to a rectangle, a square, or other polygon, as well as a circle, an ellipse, an ovoid, or other circular or semi-circular shape.

As used herein, “tissue” refers to a flexible line, part, component, structure, or flexible member of natural material, natural biological material, biomaterial, biomimetic materials, manmade material, or a combination of these either in a single structure, a composite structure, or a plurality of tissue structures that extend in parallel and/or may be woven or bonded together. In certain embodiments, a tissue may be long and thin. In certain embodiments, a tissue may be planar. In certain embodiments, a tissue may be elastic or inelastic. In a medical context, due to the flexible nature of a tissue, tissue may be referred to herein as “soft tissue.” Examples of a tissue include, but are not limited to, a thread, a suture, suture tape, a woven structure, a fibrous material, a cord, a ligament, cartilage, muscle, a ligament graft, and/or any of these in combination with each other, and the like.

As used herein, “implant” refers to a medical device manufactured to replace a missing biological structure, support a damaged biological structure, or enhance an existing biological structure. Medical implants are man-made devices. The surface of implants that contact the body may be made of, or include a biomedical material such as titanium, silicone, or apatite depending on what is the most functional. In some cases implants contain electronics, e.g. artificial pacemaker and cochlear implants. Some implants are bioactive, such as subcutaneous drug delivery devices in the form of implantable pills or drug-eluting stents. Orthopedic implants may be used to alleviate issues with bones and/or joints of a patient's body. Orthopedic implants are used to treat bone fractures, osteoarthritis, scoliosis, spinal stenosis, and chronic pain. Examples of orthopedic implants include, but are not limited to, a wide variety of pins, rods, screws, anchors, and plates used to anchor fractured bones while the bones heal or fuse together. (Search “implant (medicine)” on Wikipedia.com May 26, 2021. CC-BY-SA 3.0 Modified. Accessed Jun. 30, 2021.)

As used herein, a “fastener”, “fixation device”, or “fastener system” refers to any structure configured, designed, or engineered to join two structures. Fasteners may be made of a variety of materials including metal, plastic, composite materials, metal alloys, plastic composites, and the like. Examples of fasteners include, but are not limited to screws, rivets, bolts, nails, snaps, hook and loop, set screws, bone screws, nuts, posts, pins, thumb screws, and the like. Other examples of fasteners include, but are not limited to wires, Kirschner wires (K-wire), anchors, bone anchors, plates, bone plates, intramedullary nails or rods or pins, implants, sutures, soft sutures, soft anchors, tethers, interbody cages, fusion cages, and the like. In certain embodiments, the term fastener may refer to a fastener system that includes two or more structures configured to combine to serve as a fastener. An example of a fastener system is a rod or shaft having external threads and an opening or bore within another structure having corresponding internal threads configured to engage the external threads of the rod or shaft. In certain embodiments, the term fastener may be used with an adjective that identifies an object or structure that the fastener may be particularly configured, designed, or engineered to engage, connect to, join, contact, or couple together with one or more other structures of the same or different types. For example, a “bone fastener” may refer to an apparatus for joining or connecting one or more bones, one or more bone portions, soft tissue and a bone or bone portion, hard tissue and a bone or bone portion, an apparatus and a bone or portion of bone, or the like. In certain embodiments, a fastener may be a temporary fastener. A temporary fastener is configured to engage and serve a fastening function for a relatively short period of time. Typically, a temporary fastener is configured to be used until another procedure or operation is completed and/or until a particular event. In certain embodiments, a user may remove or disengage a temporary fastener. Alternatively, or in addition, another structure, event, or machine may cause the temporary fastener to become disengaged.

As used herein, a “body” refers to a main or central part of a structure. The body may serve as a structural component to connect, interconnect, surround, enclose, and/or protect one or more other structural components. A body may be made from a variety of materials including, but not limited to, metal, plastic, ceramic, wood, fiberglass, acrylic, carbon, biocompatible materials, biodegradable materials or the like. A body may be formed of any biocompatible materials, including but not limited to biocompatible metals such as Titanium, Titanium alloys, stainless steel alloys, cobalt-chromium steel alloys, nickel-titanium alloys, shape memory alloys such as Nitinol, biocompatible ceramics, and biocompatible polymers such as Polyether ether ketone (PEEK) or a polylactide polymer (e.g. PLLA) and/or others. In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

In one embodiment, a body may include a housing or frame or framework for a larger system, component, structure, or device. A body may include a modifier that identifies a particular function, location, orientation, operation, and/or a particular structure relating to the body. Examples of such modifiers applied to a body, include, but are not limited to, “inferior body,” “superior body,” “lateral body,” “medial body,” and the like.

As used herein, “flange” refers to a protrusion such as a protruded ridge, lip or rim, either external or internal, designed, configured, engineered, or positioned for engagement with another object and/or for attachment, or transfer of a contact force with another object (e.g., such as a flange on the end of a pipe, steam cylinder, etc., or on the lens mount of a camera); or that serves to increase strength (e.g., the flange of an iron beam such as an I-beam or a T-beam); or for stabilizing and guiding the movements of a machine or its parts (e.g., the inside flange of a rail car or tram wheel, which keep the wheels from running off the rails). (Search “flange” on Wikipedia.com May 24, 2021. CC-BY-SA 3.0 Modified. Accessed Jul. 14, 2021.)

As used herein, “feature” refers to a distinctive attribute or aspect of something. (Search “feature” on google.com. Oxford Languages, 2021. Web. 20 Apr. 2021.) A feature may include one or more modifiers that identify one or more particular functions, attributes, advantages, or operations and/or particular structures relating to the feature. Examples of such modifiers applied to a feature, include, but are not limited to, “attachment feature,” “securing feature,” “alignment feature,” “adjustment feature,” “guide feature,” “protruding feature,” “engagement feature,” “disengagement feature,” and the like.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together. “Contact” refers to an action of one object touching another object. In certain embodiments, a contact refers to a direct physical touching of the two objects. In other embodiments, a contact may refer to an indirect physical touching of the two objects. The phrase “fluid communication” refers to two features that are connected such that a fluid within one feature can pass into the other feature.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles set forth herein.

While specific embodiments and applications of the present disclosure have been illustrated and described, it is to be understood that the scope of this disclosure is not limited to the precise configuration and components disclosed herein. Various modifications, changes, and variations which will be apparent to those skilled in the art may be made in the arrangement, operation, and details of the methods and systems of the present disclosure set forth herein without departing from it spirit and scope.

What is claimed is:

1. A fastener for anchoring a tether within a body of a patient, the fastener comprising:
a one-way tissue engagement feature having an opening that receives a length of a tether and permits the length of the tether to pass through the opening in a first direction and restricts movement of the tether in a second direction opposite the first direction; and
an engagement feature configured to maintain contact between the fastener and a bone of the patient in response to tension applied to the tether in the second direction to keep the fastener in place relative to the bone.

2. The fastener of claim 1, further comprising a body that circumscribes the one-way tissue engagement feature and wherein the engagement feature extends from the body and faces at least partially toward the second direction to contact part of the bone of the patient.

3. The fastener of claim 2, wherein the body is tube shaped and the engagement feature comprises a flange circumscribing the body.

4. The fastener of claim 1, wherein:
the fastener includes a distal end and a proximal end;
the opening extends between the distal end and the proximal end; and
the engagement feature is proximal to the proximal end and the first direction starts at the distal end and continues to the proximal end.

5. The fastener of claim 1, wherein the one-way tissue engagement feature comprises a set of teeth that extend into the opening, the set of teeth configured to engage the tether within the opening such that the tether can move through the opening in the first direction and not move through the opening in the second direction.

6. The fastener of claim 5, wherein each tooth of the set of teeth comprises a base connected to an interior wall of the opening and a tip, each tooth oriented such that the tip is displaced in the first direction relative to the base.

7. The fastener of claim 6, wherein the teeth of the set of teeth are pliable and configured to bend toward the first direction as the tether is moved through the opening in the first direction.

8. The fastener of claim 7, wherein:
a tip of at least one tooth of the set of teeth comprises a tapered edge; and
the tip extends into the tether in response to a tension force on the tether in the second direction.

9. The fastener of claim 6, wherein the tip of at least one tooth of the set of teeth remains within the opening.

10. A one-way tether lock for anchoring a tissue graft within a bone tunnel, the one-way tether lock comprising:
a cylindrical body having a proximal end, a distal end, and an opening that extends from the distal end to the proximal end; and
a set of teeth that extend into the opening, the set of teeth configured to permit passage of a tissue graft from the distal end toward the proximal end and constrain passage of the tissue graft from the proximal end toward the distal end.

11. The one-way tether lock of claim 10, further comprising a stop configured to constrain movement of the one-way tether lock from a proximal end of a bone tunnel toward a distal end of the bone tunnel.

12. The one-way tether lock of claim 11, wherein the stop connects to the cylindrical body at the proximal end.

13. The one-way tether lock of claim 11, wherein the stop comprises a lip that circumscribes the cylindrical body, the lip sized to contact a surface of bone around the bone tunnel that accepts the one-way tether lock.

14. The one-way tether lock of claim 13, wherein the bone tunnel extends from a surface of a bone at first angle not perpendicular to the surface and the lip extends from the cylindrical body at a second angle that corresponds to the first angle and a first diameter of the bone tunnel is larger than a second diameter of the cylindrical body and smaller than a third diameter of the lip.

15. The one-way tether lock of claim 11, wherein the cylindrical body comprises a first diameter sized to fit within a bone tunnel configured to accept the one-way tether lock and the stop comprises part of the cylindrical body having a second diameter greater than the first diameter.

16. The one-way tether lock of claim 10, wherein teeth of the set of teeth include a base at one end and an edge at an opposite end and wherein one or more teeth of the set of teeth connect to the cylindrical body at the base and extend to point the edge towards the proximal end.

17. The one-way tether lock of claim 10, wherein teeth of the set of teeth define an initial diameter passage in the opening and the teeth are pliable and bend towards the proximal end to define a second diameter passage having a diameter greater than the initial diameter passage.

18. A method for anchoring a tissue graft within a bone tunnel of a patient, the method comprising:
forming a bone tunnel in a bone of the patient;
deploying a tissue graft within the bone tunnel;
fixing a first end of the tissue graft relative to a first end of the bone tunnel;
threading a second end of the tissue graft through an opening in a cortical graft anchor; and
sliding the tissue graft through the opening in a first direction such that teeth within the opening of the cortical graft anchor engage the tissue graft to prevent motion of the tissue graft, in a second direction opposite the first direction; and
engaging a stop of the cortical graft anchor with a second end of the bone tunnel in response to tension in the tissue graft pulling the cortical graft anchor toward the bone tunnel.

19. The method of claim 18, further comprising:
forming a cutout around a second end of the bone tunnel, the cutout sized to accept a stop of the cortical graft anchor; and
seating the stop with the cutout at the second end of the bone tunnel.

20. The method of claim 18, further comprising:
tensioning the tissue graft by pulling the tissue graft through the opening and away from the bone tunnel.
